(19) United States
(12) Patent Application Publication
(10) Pub. No.: US 2022/0039860 A1
(43) Pub. Date: Feb. 10, 2022

(54) HIGH-FREQUENCY TREATMENT TOOL

(71) Applicant: OLYMPUS CORPORATION, Tokyo (JP)

(72) Inventor: Noriaki YAMANAKA, Tokyo (JP)

(73) Assignee: OLYMPUS CORPORATION, Tokyo (JP)

(21) Appl. No.: 17/507,044

(22) Filed: Oct. 21, 2021

Related U.S. Application Data
(63) Continuation of application No. PCT/JP2019/017636, filed on Apr. 25, 2019.

(51) Int. Cl. A61B 18/14

(52) U.S. Cl. CPC A61B 18/14; A61B 2018/00077; A61B 2018/144; A61B 2018/1407

(57) ABSTRACT

A high-frequency treatment tool includes a sheath (1), a conductive wire (13) inserted into the sheath and protruding in a loop shape in a long axis direction of the sheath from a distal end of the sheath, and an operation unit (5) connected to a proximal end of the sheath to operate both end portions of the conductive wire, in which the conductive wire includes an incision unit (30) provided on a loop protruding from the distal end of the sheath and a first covering portion (31) and a second covering portion (32) disposed on both sides of the incision unit and having an insulated outer peripheral surface, and the incision unit is accommodated in the sheath asymmetrically with respect to the long axis direction by an operation of the operation unit.

This application is a continuation application based on a PCT International Application No. PCT/JP2019/017636, filed on Apr. 25, 2019. The content of the PCT International Application is incorporated herein by reference.

### BACKGROUND OF THE INVENTION

### Field of the Invention

The present invention relates to a high-frequency treatment tool.

### Description of Related Art

In the related art, in a treatment of early malignant tumors, for example, procedures such as endoscopic mucosal resection (EMR) and endoscopic submucosal dissection (ESD) are performed, which transendoscopically excise a lesion generated on a mucous membrane in a luminal organ such as a digestive tract. A high-frequency treatment tool is used as an endoscope treatment tool for excising a lesion tissue.

The high-frequency treatment tool has an incision unit which can incise a tissue by applying a high-frequency current to a distal end thereof. When the lesion tissue is incised by the high-frequency treatment tool, an operator needs to move the incision unit to a target lesion tissue and apply a high-frequency current to the incision unit in a state where the incision unit is in contact with the target lesion tissue. In addition, the operator needs to deform the incision unit in order to realize an incision shape desired by the operator.

In a high-frequency treatment tool described in Japanese Unexamined Patent Application, First Publication No. H7-23975, a conductive wire protruding from a hole on a side of a sheath can be operated by two different driving members. An operator can easily move and deform an incision unit formed by the conductive wire by operating with two different driving members.

### SUMMARY OF THE INVENTION

In order to solve the above problems, the present invention proposes the following means.

According to an aspect of the present invention, there is provided a high-frequency treatment tool including: a sheath; a conductive wire inserted into the sheath and protruding in a loop shape in a long axis direction of the sheath from a distal end of the sheath; and an operation unit connected to a proximal end of the sheath to operate both end portions of the conductive wire, in which the conductive wire includes an incision unit provided on a loop protruding from the distal end of the sheath and a first covering portion and a second covering portion disposed on both sides of the incision unit and each having an insulated outer peripheral surface, and the incision unit is accommodated in the sheath asymmetrically with respect to the long axis direction by an operation of the operation unit.

According to another aspect of the present invention, there is provided a high-frequency treatment tool including: a sheath; a conductive wire inserted into the sheath and protruding in a loop shape in a long axis direction of the sheath from a distal end of the sheath; and an operation unit connected to a proximal end of the sheath to independently operate both end portions of the conductive wire, in which the conductive wire is formed of a wire made of metal and has a first tube and a second tube disposed on both sides of an intermediate portion of the wire and covering the wire with an insulator, the wire is inserted into the first tube and the second tube in a advance/retraction movable manner, the intermediate portion of the wire which is not covered by the first tube and the second tube and in which the wire is exposed functions as an incision unit.

### BRIEF DESCRIPTION OF THE DRAWINGS

FIG. 1 is an overall view of an endoscope device used together with a high-frequency treatment tool according to a first embodiment.

FIG. 2 is a cross-sectional view of the high-frequency treatment tool in a long axis direction.

FIG. 3 is a front view when the high-frequency treatment tool is viewed from a distal end side of an incision unit.

FIG. 4 is a cross-sectional view in the long axis direction of the high-frequency treatment tool in which a first driving member and a second driving member are moved to a proximal end side.

FIG. 5 is a view showing the incision unit in which an incision shape is deformed.

FIG. 6 is a view showing the incision unit in which the incision shape is deformed.

FIG. 7 is a view showing the incision unit in which the incision shape is deformed.

FIG. 8 is a cross-sectional view of a high-frequency treatment tool according to a second embodiment in the long axis direction.

FIG. 9 is a cross-sectional view in the long axis direction of the high-frequency treatment tool in which a driving member is moved to the proximal end side.

FIG. 10 is a plan view of a modification example of the driving member of the high-frequency treatment tool.

FIG. 11 is the bottom view of a modification example of the housing of the high-frequency treatment tool.

FIG. 12 is a cross-sectional view of a high-frequency treatment tool according to a third embodiment in the long axis direction.

FIG. 13 is a side view of an operation unit of the high-frequency treatment tool.

FIG. 14 is a cross-sectional view in the long axis direction of the high-frequency treatment tool in which a shape of an incision unit is deformed.

FIG. 15 is a cross-sectional view in the long axis direction of the high-frequency treatment tool accommodating a conductive wire.

FIG. 16 is a cross-sectional view of a modification example of the conductive wire of the high-frequency treatment tool.

FIG. 17 is a perspective view of another modification example of the conductive wire of the high-frequency treatment tool.

### DETAILED DESCRIPTION OF THE INVENTION

### First Embodiment

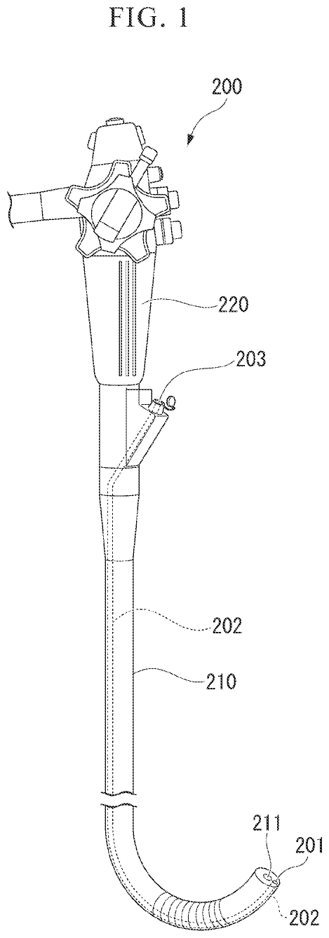

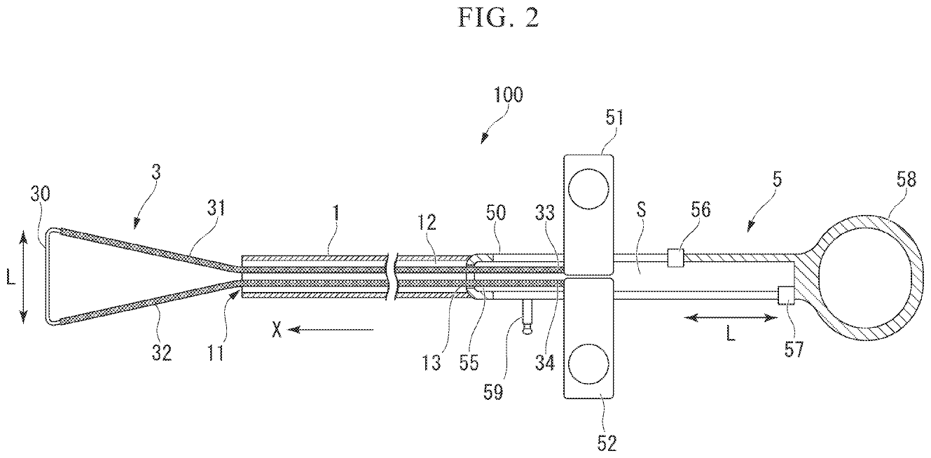

A high-frequency treatment tool 100 according to a first embodiment of the present invention will be described with reference to FIGS. 1 to 4. FIG. 1 is an overall view of an endoscope device 200 used together with the high-frequency treatment tool 100 according to the present embodiment. FIG. 2 is a cross-sectional view of the high-frequency treatment tool 100 in a long axis direction.

### [Endoscope Treatment System (Not Shown)]

An endoscope treatment system includes the high-frequency treatment tool 100 and the endoscope device 200. The high-frequency treatment tool 100 is inserted into a treatment tool channel 202 formed in an endoscope insertion portion 210 of the endoscope device 200. As shown in FIG. 1, the endoscope device 200 is a known endoscope device having the treatment tool channel 202.

The endoscope device 200 includes the endoscope insertion portion 210 inserted into a body cavity, an endoscope operation unit 220 provided at a proximal end of the endoscope insertion portion 210, and an imaging unit 211 provided at a distal end of the endoscope insertion portion 210. A distal end opening 201 of the treatment tool channel 202 is opened at a distal end portion of the endoscope insertion portion 210. The treatment tool channel 202 is a passage extending from the distal end opening 201 to the entire length of the endoscope insertion portion 210, and a proximal end portion thereof is connected to a forceps opening 203 provided in the endoscope operation unit 220.

### [High-frequency Treatment Tool 100]

As shown in FIG. 2, the high-frequency treatment tool 100 includes a jacket sheath 1, a conductive wire 3, and an operation unit 5. In the following description, a side of the operation unit 5 of the high-frequency treatment tool 100 is referred to as a proximal end side, and a side of the jacket sheath 1 opposite to the operation unit 5 in a long axis direction X is referred to as a distal end side.

The jacket sheath 1 is a long member that extends along the long axis direction X and can be inserted into the body cavity. The jacket sheath 1 is made of an insulating material, for example, a fluororesin such as polytetrafluoroethylene (PTFE). The jacket sheath 1 is flexible and is configured to be removable from the treatment tool channel 202 of the endoscope device 200 that meanders along a curved shape such as a lumen tissue in the body cavity. A lumen 12 is formed in the jacket sheath 1 over the entire length, and the jacket sheath 1 has a distal end opening 11 and a proximal end opening 13 through which the lumen 12 communicates.

The conductive wire 3 is a single metal wire, and is inserted into the jacket sheath 1 so as to be movable advance and retraction with an intermediate portion bent in a loop shape as a distal end side. Both end portions of the conductive wire 3 are attached to the operation unit 5 located on the proximal end side. The conductive wire 3 can protrude from the distal end of the jacket sheath 1 in a loop shape in the long axis direction X of the jacket sheath 1.

The conductive wire 3 has an incision unit 30 in which the wire is exposed, and a first covering portion 31 and a second covering portion 32 in which an outer peripheral surface of the wire is insulated.

The incision unit 30 is disposed in the intermediate portion of the conductive wire 3, and is disposed at a position where the conductive wire 3 protrudes when the conductive wire 3 protrudes from the distal end of the jacket sheath 1. The incision unit 30 functions as a monopolar electrode, and can incise an abutted lesion tissue by applying a high-frequency current to the incision unit 30.

The first covering portion 31 and the second covering portion 32 are disposed on both sides of the incision unit 30 in a state where the incision unit 30 is interposed therebetween. Lengths of the first covering portion 31 and the second covering portion 32 in the long axis direction are equal to each other. The first covering portion 31 is connected to the operation unit 5 at an end portion (first end portion 33) opposite to the incision unit 30. The second covering portion 32 is connected to the operation unit 5 at an end portion (second end portion 34) opposite to the incision unit 30.

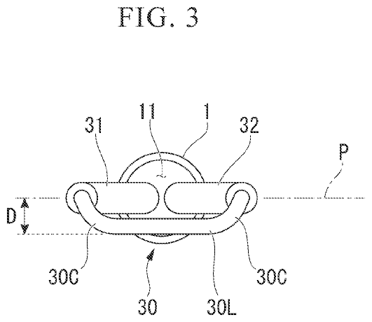

FIG. 3 is a front view when the incision unit 30 is viewed from the distal end side.

The incision unit 30 has a corner portion 30C on one end portion side connected to the first covering portion 31 and a corner portion 30C on the other end portion side connected to the second covering portion 32. A portion (hereinafter, referred to as “straight-line portion 30L”) interposed between the corner portions 30C is supported by the corner portions 30C at both ends and is formed in a straight line, and is less likely to bend when pressed against the lesion tissue.

As shown in FIG. 3, the incision unit 30 can be disposed bilaterally symmetrically with respect to the long axis direction X of the jacket sheath 1 when the conductive wire 3 protrudes from the distal end of the jacket sheath 1. Therefore, the incision unit 30 easily incises the lesion tissue located in front of the jacket sheath 1.

As shown in FIG. 3, the incision unit 30 has a displacement D in a direction perpendicular to a loop surface P formed when the conductive wire 3 protrudes from the distal end of the jacket sheath 1. The incision unit 30 protrudes in the vertical direction with respect to the loop surface P, and the incision unit 30 is easily pressed against the lesion tissue.

As shown in FIG. 2, the operation unit 5 includes a housing 50 connected to the proximal end portion of the jacket sheath 1, a first driving member 51, a second driving member 52, a handle 58, and a power supply connector 59.

As shown in FIG. 2, the housing 50 has an internal space S into which the conductive wire 3 can be inserted. A distal end opening 55 of the internal space S communicates with the proximal end opening 13 of the jacket sheath 1. The conductive wire 3 passes through the proximal end opening 13 of the jacket sheath 1 and the distal end opening 55 of the internal space S and extends to the internal space S.

The first driving member 51 is attached to the housing 50 so as to be slidable in the long axis direction X of the jacket sheath 1. The first end portion 33 of the conductive wire 3 is connected to the first driving member 51. When an operator moves the first driving member 51 advance and retraction relatively with respect to the housing 50, the first covering portion 31 of the conductive wire 3 moves advance and retraction with respect to the jacket sheath 1.

The second driving member 52 is attached to the housing 50 so as to be slidable in the long axis direction X of the jacket sheath 1. The first driving member 51 and the second driving member 52 can be operated independently of each other with respect to the housing 50. The second end portion 34 of the conductive wire 3 is connected to the second driving member 52. When the operator moves the second driving member 52 advance and retraction relatively with respect to the housing 50, the second covering portion 32 of the conductive wire 3 moves advance and retraction with respect to the jacket sheath 1.

As shown in FIG. 2, the housing 50 has a first stopper 56 and a second stopper 57. The first stopper 56 and the second stopper 57 are fixed to the housing 50. The second stopper 57 is fixed to the housing 50 on the proximal end side by a distance L as compared with the first stopper 56.

The first stopper 56 limits an operating range of the first driving member 51 toward the proximal end side. The first stopper 56 comes into contact with the first driving member 51, and thus, limits a movement of the first driving member 51 from a position where the first stopper 56 is disposed to the proximal end side.

The second stopper 57 limits an operating range of the second driving member 52 toward the proximal end side. The second stopper 57 comes into contact with the second driving member 52, and thus, limits a movement of the second driving member 52 from a position where the second stopper 57 is disposed to the proximal end side.

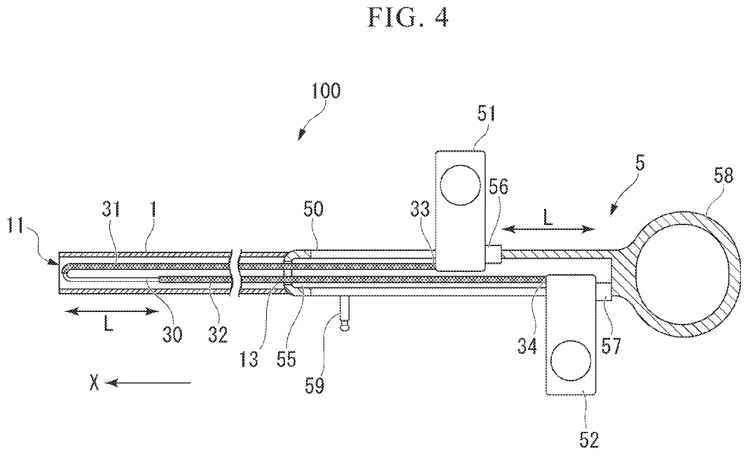

FIG. 4 is a cross-sectional view in the long axis direction of the high-frequency treatment tool 100 in which the first driving member 51 and the second driving member 52 are moved to the proximal end side. When the first driving member 51 is moved toward the proximal end side until the first driving member 51 comes into contact with the first stopper 56, the first covering portion 31 is completely accommodated in the lumen 12 of the jacket sheath 1.

When the first driving member 51 is moved to the proximal end side until the second driving member 52 comes into contact with the second stopper 57, the second covering portion 32 and the incision unit 30 extend straight and are completely accommodated in the lumen 12 of the jacket sheath 1. Therefore, the incision unit 30 is accommodated in the lumen 12 of the jacket sheath 1 in a state where the straight-line portion 30L is not bent.

A wire length of the incision unit 30 is L. Meanwhile, the second stopper 57 is disposed on the proximal end side by the distance L as compared with the first stopper 56. Therefore, when the first stopper 56 and the second stopper 57 are moved to the same position in the long axis direction X, as shown in FIG. 2, the incision unit 30 is disposed bilaterally symmetrically with respect to the long axis direction X of the jacket sheath 1.

The handle 58 is a member fixed to the housing 50. The operator holds the first driving member 51, the second driving member 52, and the handle 58 to perform a procedure.

The power supply connector 59 can be connected to a high-frequency power supply device (not shown), and is electrically and physically connected to the proximal end portion of the conductive wire 3. The power supply connector 59 can supply a high-frequency current supplied from the high-frequency power supply device to the conductive wire 3.

### [Operation of Endoscope Treatment System (Not Shown)]

Next, an operation of the endoscope treatment system will be described. The operation of the endoscope treatment system will be described by taking as an example a procedure of incising a lesion portion (early cancer or the like) P formed in a large intestine using the endoscope treatment system.

As a preparatory work, the operator identifies the lesion portion P by a known method and bulges the lesion portion P. Specifically, the operator inserts the endoscope insertion portion 210 of the endoscope device 200 into the large intestine, and identifies the lesion portion P while observing the image obtained by the imaging unit 211 of the endoscope. Next, the operator inserts a known submucosal local injection needle (not shown) into the treatment tool channel 202 of the endoscope insertion portion 210, and injects a liquid for local injection (local injection liquid) between the lesion portion P and a muscular layer W3 by the submucosal local injection needle to bulge the lesion portion P. After the operator injects the local injection liquid, the operator removes the submucosal local injection needle from the treatment tool channel 202.

The operator inserts the high-frequency treatment tool 100 into the treatment tool channel 202, and protrudes the distal end portion of the jacket sheath 1 from the distal end opening 201 of the endoscope insertion portion 210. When the high-frequency treatment tool 100 is inserted into the treatment tool channel 202, the conductive wire 3 is accommodated inside the lumen 12 of the jacket sheath 1. The operator protrudes the distal end portion of the jacket sheath 1 to the vicinity of the lesion portion P while checking an endoscopic image.

The operator moves the first driving member 51 and the second driving member 52 of the operation unit 5 forward relatively with respect to the housing 50, and thus, the conductive wire 3 protrudes from the distal end opening 11 of the jacket sheath 1. The first stopper 56 and the second stopper 57 are moved to the same position in the long axis direction X, and the incision unit 30 is disposed bilaterally symmetrically with respect to the long axis direction X of the jacket sheath 1. The operator moves the first covering portion 31 or the second covering portion 32 advance and retraction to deform the incision unit 30 into an incision shape desired by the operator.

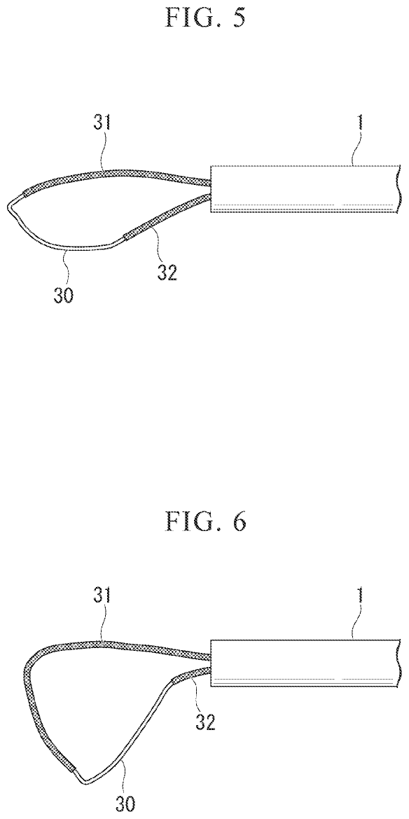

FIGS. 5 to 7 are views showing the incision unit 30 in which the incision shape is deformed.

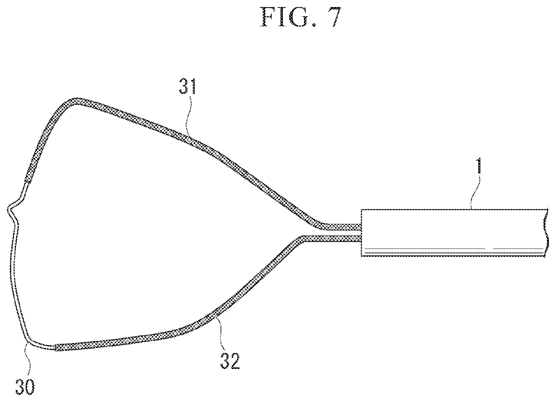

As shown in FIG. 5, the operator moves the second covering portion 32 rearward with respect to the first covering portion 31, and thus, the incision unit 30 can be deformed in a shape that is not bilaterally symmetrical with respect to the long axis direction X of the jacket sheath 1. The operator further moves the first covering portion 31 forward and moves the second covering portion 32 rearward, and thus, as shown in FIG. 6, the incision unit 30 can be disposed at a position inclined with respect to the long axis direction X of the jacket sheath 1. Further, as shown in FIG. 7, the operator may move the first covering portion 31 and the second covering portion 32 forward, and push the incision unit 30 forward with respect to the long axis direction X of the jacket sheath 1 to dispose the incision unit 30.

The operator moves the endoscope insertion portion 210 or the jacket sheath 1 advance and retraction as appropriate, and presses the incision unit 30 against the lesion portion P. Since the straight-line portion 30L is less likely to bend due to the corner portions 30C at both ends, the operator can easily press the straight-line portion 30L against the lesion portion P. Further, when the straight-line portion 30L is disposed so as to face a direction perpendicular to the long axis direction X of the jacket sheath 1, the operator moves the endoscope insertion portion 210 or the jacket sheath 1 advance and retraction, and thus, the straight-line portion 30L can be easily pressed against a certain lesion portion P located in front of the straight-line portion 30L.

The operator supplies a high-frequency current to the power supply connector 59, and causes the high-frequency current to flow from the conductive wire 3 to a mucosal layer W1 of the lesion portion P to incise the lesion portion P.

After the operator incises the lesion portion P, the operator moves the first driving member 51 and the second driving member 52 toward the proximal end side, and accommodates the conductive wire 3 in the lumen 12 of the jacket sheath 1. The second driving member 52 is first moved to the proximal end side so that the straight-line portion 30L of the incision unit 30 becomes horizontal with respect to the long axis direction X of the jacket sheath 1, and then the first driving member 51 and the second driving member 52 is moved to the proximal end side at the same time. By accommodating the conductive wire 3 in this way, it is possible to prevent the straight-line portion 30L from bending.

According to the high-frequency treatment tool 100 of the present embodiment, the first driving member 51 and the second driving member 52 can be operated independently of each other to easily realize the incision shape of the incision unit 30 desired by the operator.

According to the high-frequency treatment tool 100 according to the present embodiment, when the first stopper 56 and the second stopper 57 are moved to the same position in the long axis direction X, since the incision unit 30 is disposed bilaterally symmetrically with respect to the long axis direction X of the jacket sheath 1, it is easy to operate the incision unit 30.

According to the high-frequency treatment tool 100 of the present embodiment, it is possible to prevent the incision unit 30 from bending near the center when the conductive wire 3 is accommodated. Therefore, even when the conductive wire 3 repeatedly protrudes and is accommodated, the shape in which the straight-line portion 30L extends in a straight line can be maintained at the time of protrusion.

As described above, the first embodiment of the present invention is described in detail with reference to the drawings. However, specific configurations are not limited to the embodiment, and include a design modification or the like within a scope which does not depart from the gist of the present invention. In addition, components shown in the above-described embodiment and modification examples shown below can be appropriately combined and configured.

### Second Embodiment

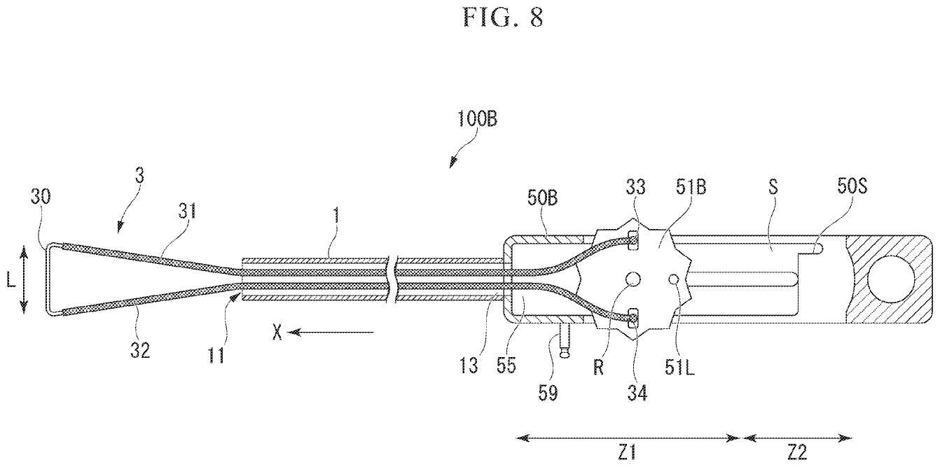

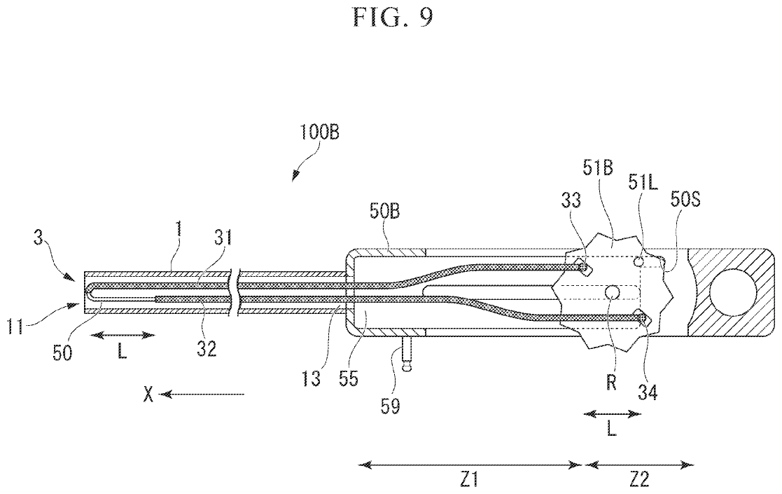

A high-frequency treatment tool 100B of a second embodiment of the present invention will be described with reference to FIGS. 8 and 9. In the following description, the same components as those already described are denoted by the same reference numerals, and repeated descriptions will be omitted. In the high-frequency treatment tool 100B, a first driving member and a second driving member are different from those of the first embodiment.

### [High-Frequency Treatment Tool 100B]

FIG. 8 is a cross-sectional view of the high-frequency treatment tool 100B in the long axis direction.

As shown in FIG. 8, the high-frequency treatment tool 100B includes the jacket sheath 1, the conductive wire 3, and an operation unit 5B. In the following description, a side of the operation unit 5B of the high-frequency treatment tool 100B is referred to as a proximal end side, and a side of the jacket sheath 1 opposite to the operation unit 5B in the long axis direction X is referred to as a distal end side.

As shown in FIG. 8, the operation unit 5B includes a housing 50B connected to the proximal end portion of the jacket sheath 1, a driving member 51B, the handle 58, and the power supply connector 59.

The housing 50B has an internal space S into which the conductive wire 3 can be inserted. A distal end opening 55 of the internal space S communicates with the proximal end opening 13 of the jacket sheath 1. The conductive wire 3 passes through the proximal end opening 13 of the jacket sheath 1 and the distal end opening 55 of the internal space S and extends to the internal space S.

The driving member 51B is attached to the housing 50 so as to be slidable in the long axis direction X of the jacket sheath 1. Further, the driving member 51B is rotatably attached to the housing 50 about a rotation shaft member R perpendicular to the long axis direction X. The first end portion 33 and the second end portion 34 of the conductive wire 3 are connected to the driving member 51B. When the operator moves the driving member 51B advance and retraction relatively with respect to the housing 50B, the first covering portion 31 and the second covering portion 32 of the conductive wire 3 move advance and retraction with respect to the jacket sheath 1.

As shown in FIG. 8, the first end portion 33 and the second end portion 34 of the conductive wire 3 are attached to the driving member 51B on both sides of the rotation shaft member R in a state where the rotation shaft member R is interposed therebetween. By rotating the driving member 51B about the rotation shaft member R, the operator can move one of the first covering portion 31 and the second covering portion 32 forward and move the other rearward at the same time.

The driving member 51B has a convex rib 51L. The housing 50B has a slit 50S through which the rib 51L can pass in a second region Z2 on the proximal end side. When the driving member 51B is in a first region Z1 on the distal end side, the driving member 51B can slide and move in the long axis direction X regardless of the position of the rib 51L. Meanwhile, when the rib 51L is not disposed at the position where the rib 51L can pass through the slit 50S, the driving member 51B cannot pass through the second region Z2 on the proximal end side.

FIG. 9 is a cross-sectional view in the long axis direction of the high-frequency treatment tool 100B in which the driving member 51B is moved to the proximal end side.

When the driving member 51B rotates about the rotation shaft member R and the second covering portion 32 moves to the proximal end side of the first covering portion 31 by the wire length L of the incision unit 30, the rib 51L is disposed at the position where the rib 51L can pass through the slit 50S. By sliding the driving member 51B in this state toward the proximal end side, the rib 51L passes through the slit 50S and the driving member 51B moves to the second region Z2.

By moving the driving member 51B to the proximal end side of the second region Z2, the conductive wire 3 is accommodated in the lumen 12 of the jacket sheath 1. The second covering portion 32 and the incision unit 30 extend straight and are completely accommodated in the lumen 12 of the jacket sheath 1. Therefore, the incision unit 30 is accommodated in the lumen 12 of the jacket sheath 1 in a state where the straight-line portion 30L is not bent. The incision unit 30 is accommodated in the lumen 12 asymmetrically with respect to the long axis direction X.

According to the high-frequency treatment tool 100B of the present embodiment, the incision shape of the incision unit 30 desired by the operator can be easily realized by operating only the driving member 51B. It is possible to prevent the incision unit 30 from bending near the center when the conductive wire 3 is accommodated. Therefore, even when the conductive wire 3 repeatedly protrudes and is accommodated, the shape in which the straight-line portion 30L extends in a straight line can be maintained at the time of protrusion.

As described above, the second embodiment of the present invention is described in detail with reference to the drawings. However, specific configurations are not limited to the embodiment, and include a design modification or the like within a scope which does not depart from the gist of the present invention. In addition, components shown in the above-described embodiment and modification examples shown below can be appropriately combined and configured.

### Modification Example 1

In the above embodiment, the driving member 51B can slide and move in the long axis direction X without resistance to the housing 50B, and can rotate about the rotation shaft member R without resistance. However, the aspects of the driving member and the housing are not limited to this. The driving member and the housing may have a structure in which a frictional force changes stepwise in the sliding movement and the rotational movement.

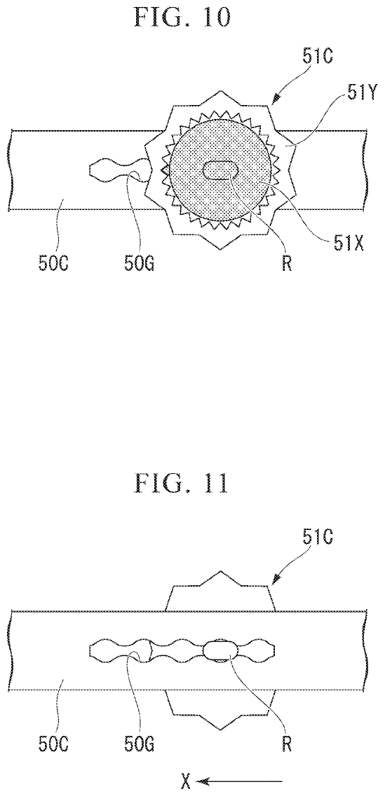

FIG. 10 is a plan view of a driving member 51C, which is a modification example of the driving member.

The driving member 51C has a fixed portion 51X fixed to the rotation shaft member R so as not to rotate, and a rotating portion 51Y rotating around the fixed portion 51X. The first covering portion 31 and the second covering portion 32 are fixed to the rotating portion 51Y. Uneven portions are formed on an outer peripheral portion of the fixed portion 51X, and uneven portions are formed on an inner peripheral surface of the rotating portion 51Y. The rotating portion 51Y rotates in a stepped manner by engaging the uneven portions with respect to the fixed portion 51X.

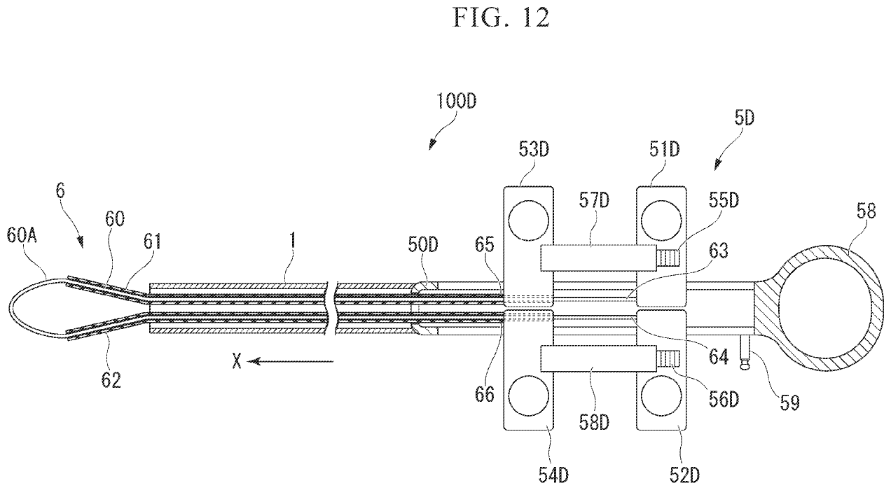

FIG. 11 is a bottom view of a housing 50C, which is a modification example of the housing.

A bottom surface of the housing 50C has a groove portion 50G that supports the rotation shaft member R so as to be slidable in the long axis direction X. A side surface of the groove portion 50G in contact with the rotation shaft member R is formed in an uneven shape. The rotation shaft member R and the driving member 51C slide and move in a stepped manner with respect to the housing 50C.

When the driving member and the housing have a structure in which the frictional force changes stepwise in the sliding movement and the rotational movement as described above, the operator can easily maintain the position and shape of the incision unit 30 even when the driving member and the housing are released from hands of the operation.

### Third Embodiment

A high-frequency treatment tool 100D according to a third embodiment of the present invention will be described with reference to FIGS. 12 to 15. In the following description, the same components as those already described are denoted by the same reference numerals, and repeated descriptions will be omitted. In the high-frequency treatment tool 100D, a conductive wire, a first driving member, and a second driving member are different from those of the first embodiment.

### [High-Frequency Treatment Tool 100D]

FIG. 12 is a cross-sectional view of a high-frequency treatment tool 100D according to a third embodiment in the long axis direction.

As shown in FIG. 12, the high-frequency treatment tool 100D includes the jacket sheath 1, a conductive wire 6, and an operation unit 5D.

The conductive wire 6 has a single metal wire 60 and a first tube 61 and a second tube 62 in which the wire 60 is covered with an insulator. The wire 60 is inserted into the first tube 61 and the second tube 62 in a advance and retraction movable manner. The first tube 61 and the second tube 62 are disposed on both sides of an intermediate portion of the wire 60 in a state where the intermediate portion is interposed therebetween.

The conductive wire 6 is inserted into the jacket sheath 1 in a advance and retraction movable manner with the intermediate portion bent in a loop shape as the distal end side. The conductive wire 6 can protrude from the distal end of the jacket sheath 1 in a loop shape in the long axis direction X of the jacket sheath 1. In the conductive wires 6 protruding from the jacket sheath 1, an intermediate portion where the wire 60 is exposed without being covered by the first tube 61 or the second tube 62 functions as an incision unit 60A. The incision unit 60A functions as a monopolar electrode, and can incise an abutted lesion tissue by applying a high-frequency current to the incision unit 60A.

As shown in FIG. 12, the operation unit 5D includes a housing 50D connected to the proximal end portion of the jacket sheath 1, a first driving member 51D, a second driving member 52D, a third driving member 53D, a fourth driving member 54D, a handle 58, and a power supply connector 59.

As shown in FIG. 12, the housing 50D has an internal space S into which the conductive wire 3 can be inserted. A distal end opening 55 of the internal space S communicates with the proximal end opening 13 of the jacket sheath 1. The conductive wire 6 passes through the proximal end opening 13 of the jacket sheath 1 and the distal end opening 55 of the internal space S and extends to the internal space S.

The first driving member 51D is attached to the housing 50D so as to be slidable in the long axis direction X of the jacket sheath 1. A first end portion 63 of the wire 60 is connected to the first driving member 51D. When the operator moves the first driving member 51D advance and retraction relatively with respect to the housing 50D, the wire 60 moves advance and retraction with respect to the jacket sheath 1.

The second driving member 52D is attached to the housing 50D so as to be slidable in the long axis direction X of the jacket sheath 1. The first driving member 51D and the second driving member 52D can be operated independently of each other with respect to the housing 50D. A second end portion 64 of the wire 60 is connected to the second driving member 52D. When the operator moves the second driving member 52D advance and retraction relatively with respect to the housing 50D, the wire 60 moves advance and retraction with respect to the jacket sheath 1.

The third driving member 53D is attached to the housing 50D so as to be slidable in the same direction as the first driving member 51D. The third driving member 53D is arranged side by side with the first driving member 51D in the long axis direction X. A proximal end portion 65 of the first tube 61 is connected to the third driving member 53D. When the operator moves the third driving member 53D advance and retraction relatively with respect to the housing 50D, the first tube 61 moves advance and retraction with respect to the jacket sheath 1.

The fourth driving member 54D is attached to the housing 50D so as to be slidable in the same direction as the second driving member 52D. The fourth driving member 54D is arranged side by side with the second driving member 52D in the long axis direction X. A proximal end portion 66 of the second tube 62 is connected to the fourth driving member 54D. When the operator moves the fourth driving member 54D advance and retraction relatively with respect to the housing 50D, the second tube 62 moves advance and retraction with respect to the jacket sheath 1.

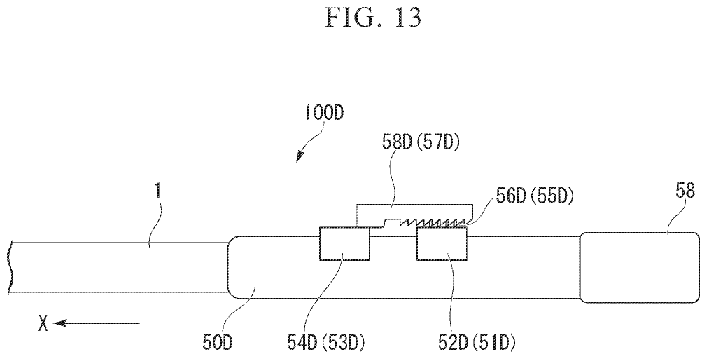

FIG. 13 is a side view of the operation unit 5D.

The first driving member 51D has a first engagement portion 55D that engages with the third driving member 53D in a disengageable manner. Meanwhile, the third driving member 53D has a first engaged portion 57D that engages with the first engagement portion 55D in a disengageable manner. The first engagement portion 55D and the first engaged portion 57D engage with each other on a surface formed in an uneven shape. Therefore, when any one of the first driving member 51D and the third driving member 53D moves, the other also moves in conjunction with each other. Meanwhile, by moving the other of the first driving member 51D and the third driving member 53D in a state where one thereof is fixed, the engagement can be temporarily released and the other can be relatively moved.

The second driving member 52D has a second engagement portion 56D that engages with the fourth driving member 54D in a disengageable manner. Meanwhile, the fourth driving member MD has a second engaged portion 58D that engages with the second engagement portion 56D in a disengageable manner. The second engagement portion 56D and the second engaged portion 58D engage with each other on a surface formed in an uneven shape. Therefore, when any one of the second driving member 52D and the fourth driving member 54D moves, the other also moves in conjunction with each other. Meanwhile, by moving the other of the second driving member 52D and the fourth driving member 54D in a state where one thereof is fixed, the engagement can be temporarily released and the other can be relatively moved.

### [Operation of High-Frequency Treatment Tool 100D]

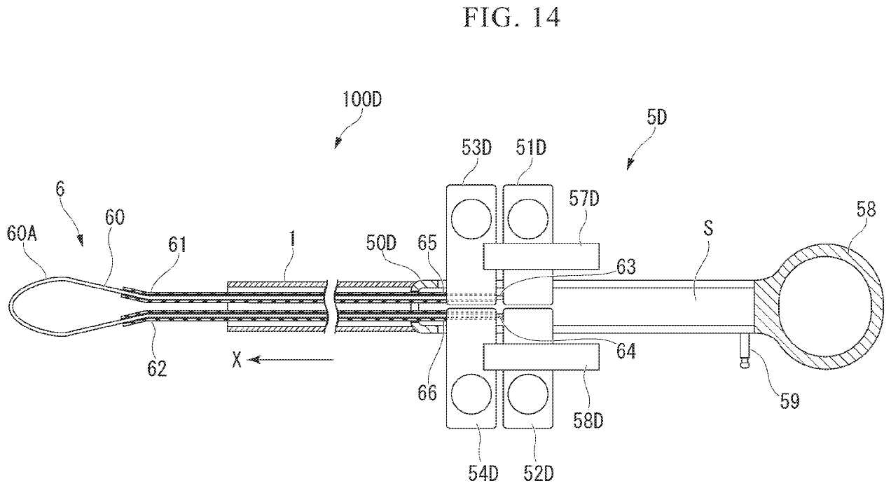

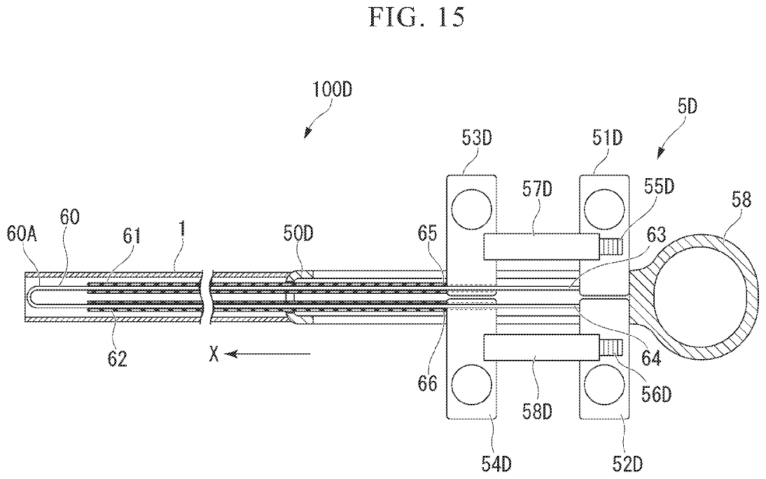

Next, an operation of the high-frequency treatment tool 100D of the present embodiment will be described. FIG. 14 is a cross-sectional view in the long axis direction of the high-frequency treatment tool 100D in which the shape of the incision unit 60A is deformed. FIG. 15 is a cross-sectional view in the long axis direction of the high-frequency treatment tool 100D accommodating the conductive wire 6.

When the first driving member 51D is moved to the distal end side in a state where the third driving member 53D is fixed and not moved, as shown in FIG. 14, the wire 60 inserted into the first tube 61 moves to the distal end side. Further, when the second driving member 52D is moved to the distal end side in a state where the fourth driving member 54D is fixed and not moved, as shown in FIG. 14, the wire 60 through which the second tube 62 is inserted moves to the distal end side. As a result, the length of the incision unit 60A, which is a portion where the wire 60 is exposed without being covered with the first tube 61 or the second tube 62, becomes longer. The operator can easily realize the incision shape of the incision unit 60A desired by the operator by moving the first driving member 51D and the second driving member 52D.

After the operator obtains the incision shape of the incision unit 60A desired by the operator, the operator interlocks the first driving member 51D and the third driving member 53D, and interlocks the second driving member 52D and the fourth driving member 54D to perform the movement, and thus, the operator can easily move the position of the incision unit 60A while maintaining the incision shape of the incision unit 60A.

After the operator incises the lesion portion P, as shown in FIG. 15, the operator moves the first driving member 51D, the second driving member 52D, the third driving member 53D, and the fourth driving member 54D to the proximal end side to accommodate the conductive wire 6 in the lumen 12 of the jacket sheath 1. By forcibly moving the first driving member 51D and the second driving member 52D toward the proximal end side, the conductive wire 6 can be smoothly accommodated in the lumen 12 of the jacket sheath 1 as shown in FIG. 15.

According to the high-frequency treatment tool 100D of the present embodiment, the first driving member 51D, the second driving member 52D, the third driving member 53D, and the fourth driving member 54D are operated independently of each other, and thus, the incision shape of the incision unit 30 desired by the operator can be easily realized.

According to the high-frequency treatment tool 10D0 of the present embodiment, the first driving member 51D and the third driving member 53D are interlocked with each other and the second driving member 52D and the fourth driving member 54D are interlocked with each other to perform the operation. Therefore, even in the operation unit 5D having a high degree of freedom of operation, the position of the incision unit 60A can be easily moved while maintaining the incision shape of the incision unit 60A.

As described above, the third embodiment of the present invention is described in detail with reference to the drawings. However, specific configurations are not limited to the embodiment, and include a design modification or the like within a scope which does not depart from the gist of the present invention. In addition, components shown in the above-described embodiment and modification examples shown below can be appropriately combined and configured.

### Modification Example 2

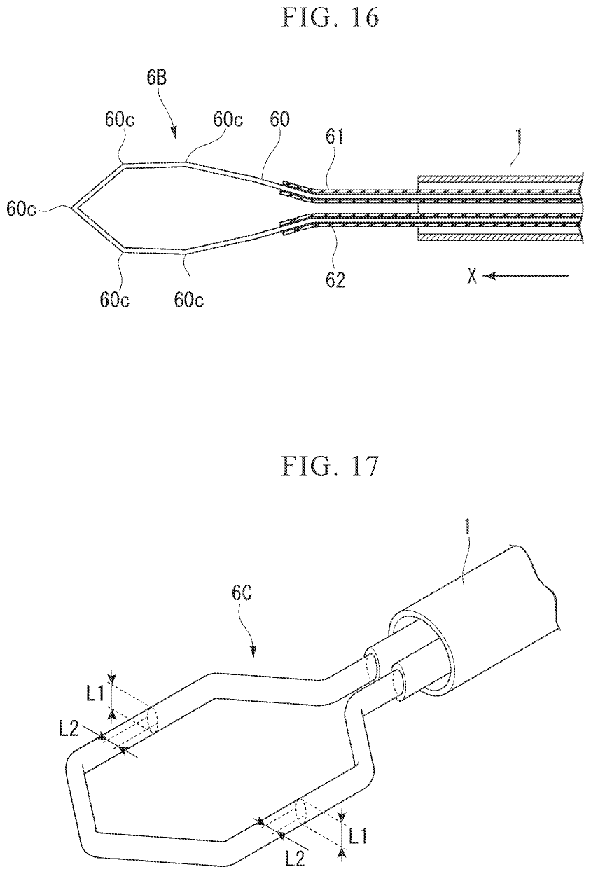

In the above embodiment, the conductive wire 6 is a metal wire having no corner portions, but the aspect of the conductive wire is not limited to this. FIG. 16 is a cross-sectional view of a conductive wire 6B, which is a modification example of the conductive wire, in the long axis direction. The conductive wire 6B has five corner portions 60C. A portion interposed between the corner portions 60C is supported by the corner portions 60C at both ends and is formed in a straight line, and is less likely to bend when pressed against the lesion tissue. Further, the operator may use only the linear portion of the conductive wire 6B interposed between the two corner portions 60C as the incision unit as in the incision unit 30 of the first embodiment. Even in that case, the operator can independently operate the first driving member 51D, the second driving member 52D, the third driving member 53D, and the fourth driving member 54D to easily change a shape and a disposition direction of the formed incision unit.

### Modification Example 3

In the above embodiment, the conductive wire 6 is a single metal wire, but the aspect of the conductive wire is not limited to this. The conductive wire may be a stranded metal wire. Further, as in a conductive wire 6C which is a modification example of the conductive wire shown in FIG. 17, at least in the incision unit where the incision is performed, a length L1 in the vertical direction with respect to a loop surface formed when the conductive wire 6C protrudes from the distal end of the jacket sheath 1 may be longer than a length L2 in a horizontal direction. The conductive wire 6C has high rigidity in the pressing direction (vertical direction) and is not easily twisted. Therefore, the conductive wire 6C can be pressed to realize a stable incision.

### Modification Example 4

In the above embodiment, the first driving member 51D and the second driving member 52D can be operated independently, but the aspects of the first driving member and the second driving member are not limited to this. The first driving member and the second driving member may be connected and operated as one. A degree of freedom of the operation unit is slightly reduced, but on the other hand, the operation of the conductive wire 6 becomes easy. Similarly, the third driving member and the fourth driving member may be connected and operated as one.

### Modification Example 5

In the above embodiment, the first tube 61 and the second tube 62 are separate tubes, but the aspects of the first tube and the second tube are not limited to this. The first tube and the second tube may be integrated in the jacket sheath 1. When the third driving member and the fourth driving member are connected and can operate as one, the integrated first tube and second tube can be moved advance and retraction together, and the operation becomes easy. Further, it is easy to maintain the shape of the incision unit 60A in a bilaterally symmetrical shape.

What is claimed is:

1. A high-frequency treatment tool comprising:
a sheath;
a conductive wire inserted into the sheath and protruding in a loop shape in a long axis direction of the sheath from a distal end of the sheath; and
an operation unit connected to a proximal end of the sheath, the operation unit being configured to operate both end portions of the conductive wire,
wherein the conductive wire includes an incision unit provided on a loop protruding from the distal end of the sheath and a first covering portion and a second covering portion disposed on both sides of the incision unit and having an insulated outer peripheral surface,
the incision unit includes two corner portions,
in the incision unit, a region formed between the corner portions is deformable linearly, and
in the incision unit, the region formed between the corner portions is accommodated without creases when accommodated in the sheath.

2. The high-frequency treatment tool according to claim 1, wherein lengths of the first covering portion and the second covering portion are equal to each other.

3. The high-frequency treatment tool according to claim 2, wherein the conductive wire is asymmetrically accommodated in the sheath so that one side of the conductive wire from an apex of the loop closed when being accommodated in the sheath is the first covering portion and the other side thereof is the incision unit and the second covering portion.

4. The high-frequency treatment tool according to claim 1, wherein the incision unit has a displacement in a direction perpendicular to a loop surface formed when the conductive wire protrudes from the distal end of the sheath.

5. The high-frequency treatment tool according to claim 1,
wherein the operation unit includes a housing, and a first driving member and a second driving member slidable independently of each other with respect to the housing, and
an end portion of the first covering portion is connected to the first driving member and an end portion of the second covering portion is connected to the second driving member.

6. The high-frequency treatment tool according to claim 5,
wherein the housing includes a first stopper which limits an operating range of the first driving member toward a proximal end side, and a second stopper which limits an operating range of the second driving member toward the proximal end side, and the second stopper is fixed to the housing at the proximal end side by a wire length of the incision unit as compared with the first stopper.

7. The high-frequency treatment tool according to claim 1,
wherein the operation unit includes a housing and a driving member which is slidably and rotatably disposed with respect to the housing and to which both end portions of the conductive wire are connected, and
one of the first covering portion and the second covering portion moves forward and the other moves rearward at the same time by a rotation of the driving member.

8. The high-frequency treatment tool according to claim 7,
wherein the operation unit includes a first region slidable with respect to the housing regardless of a rotation angle of the driving member, and a second region slidable with respect to the housing when the rotation angle of the driving member is a predetermined angle.

9. The high-frequency treatment tool according to claim 7,
wherein the driving member and the housing have a structure in which a frictional force changes stepwise in a sliding movement and a rotational movement of the driving member with respect to the housing.

10. A high-frequency treatment tool comprising:
a sheath;
a conductive wire inserted into the sheath, protruding in a loop shape in a long axis direction of the sheath from a distal end of the sheath, formed of a wire made of metal, and having a first tube and a second tube disposed on both sides of an intermediate portion of the wire to cover the wire with an insulator, in which the wire is inserted into the first tube and the second tube in a advance/retraction movable manner, and the wire includes a conductive wire in which the intermediate portion which is not covered by the first tube and the second tube and in which the wire is exposed functions as an incision unit; and
an operation unit connected to a proximal end of the sheath, the operation unit being configured to independently operate both end portions of the conductive wire,
wherein the operation unit includes
a housing,
a first driving member slidably supported with respect to the housing and connected to a first end portion of the wire,
a second driving member slidably supported with respect to the housing and connected to a second end portion of the wire,
a third driving member slidable in the same direction as that of the first driving member with respect to the housing and to which the first tube is connected, and
a fourth driving member slidable in the same direction as that of the second driving member with respect to the housing and to which the second tube is connected.

11. The high-frequency treatment tool according to claim 10,
wherein the incision unit has corner portions at both ends.

12. The high-frequency treatment tool according to claim 10 or 11,
wherein a length of the conductive wire in a vertical direction with respect to a loop surface formed when the conductive wire protrudes from the distal end of the sheath is longer than a length thereof in a horizontal direction, at least in the incision unit.

13. The high-frequency treatment tool according to claim 10,
wherein the first driving member and the third driving member engage with each other in a disengageable manner, and
the second driving member and the fourth driving member engage with each other in a disengageable manner.
